(19) United States
(12) Patent Application Publication
(10) Pub. No.: US 2023/0058253 A1
(43) Pub. Date: Feb. 23, 2023

(54) TREATMENT INSTRUMENT

(71) Applicant: Olympus Medical Systems Corp., Tokyo (JP)

(72) Inventors: Ojiro KITAMURA, Tokyo (JP); Tatsuya SUZUKI, Tokyo (JP); Hiroshi MINAMI, Tokyo (JP)

(73) Assignee: Olympus Medical Systems Corp., Tokyo (JP)

(21) Appl. No.: 17/885,618

(22) Filed: Aug. 11, 2022

Related U.S. Application Data
(60) Provisional application No. 63/235,193, filed on Aug. 20, 2021.

(51) Int. Cl. A61B 17/29; A61B 17/32

(52) U.S. Cl. CPC A61B 17/29; A61B 17/320092; A61B 2017/320095

(57) ABSTRACT

Treatment instrument includes a housing having a fixed handle to be grasped by a user, an end effector for grasping biological tissue by opening and closing, a movable handle which is rotatable with respect to the housing and moves in a direction toward or away from the fixed handle by rotating with respect to the housing, an opening and closing mechanism for opening and closing the end effector according to the rotation of the movable handle, a first fulcrum serving as a first rotation shaft for rotating the movable handle, a first slot through which the first fulcrum is inserted, a second fulcrum provided at a position different from the first fulcrum and serving as a second rotation shaft for rotating the movable handle, and a second slot through which the second fulcrum is inserted.

### RELATED APPLICATION DATA

This application is based on and claims priority under 37 U.S.C. § 119 to U.S. Provisional Application No. 63/235,193 filed on Aug. 20, 2021, the entire contents of each of these applications are incorporated herein by reference.

### FIELD OF THE INVENTION

The present invention relates to a treatment instrument.

### DESCRIPTION OF THE RELATED ART

Conventionally, there has been known a treatment instrument which treats a site of interest by imparting treatment energy to a site (hereinafter, described as a target site) to be treated in a biological tissue (see, for example, Patent Document 1). The treatment instrument described in Patent Document 1 includes a movable handle which moves in a direction of approaching or separating from a fixed handle provided in the housing by rotating with respect to the housing, and an opening/closing mechanism which opens/closes the end effector according to the rotation of the movable handle. Here, the movable handle includes an operation unit for receiving a rotation operation by the user, and a transmission unit for transmitting a force to the opening and closing mechanism in response to rotation. Then, in the treatment tool described in Patent Document 1, a leverage ratio which is a ratio of a second distance between the rotation axis and the operation portion to a first distance between the rotation axis and the transmission portion for rotating the movable handle is increased. Thus, the movable handle is rotated to reduce the amount of operation force when grasping the biological tissue by the end effector.

### Prior Art Documents

Patent Document 1: U.S. Pat. No. 9,492,225.

### SUMMARY OF THE INVENTION

### Problem to be Solved by the Invention

However, in the treatment instrument described in Patent Document 1, since the leverage ratio is increased, the distance that the movable handle moves when the movable handle is rotated and operated increases, e.g., an amount of the stroke length of the movable handle increases. Therefore, a technique that can improve operability is desired in which while reducing the amount of operation force to the movable handle is reduced and the amount of the stroke length of the movable handle is reduced.

In view of the above, it is an object of the present invention to provide a treatment instrument capable of improving operability.

### Means for Solving the Problem

In order to solve the above problems and achieve the purpose, a treatment tool according to the present invention includes a housing having a fixed handle configured to be grasped by a user, an end effector configured to grasp biological tissue by opening and closing, a movable handle which is rotatable with respect to the housing and moves in a first direction toward the fixed handle or in a second direction away from the fixed handle by rotating with respect to the housing, an opening and closing mechanism configured to open and close the end effector according to the rotation of the movable handle, a first fulcrum serving as a first axis of rotation about which the movable handle rotates in a first rotation segment, a first slot through which the first fulcrum is inserted, a second fulcrum provided at a position different from the first fulcrum and serving as a second axis of rotation about which the movable handle rotates in a second rotation segment, and a second slot through which the second fulcrum is inserted.

Rotation of the movable handle during the first rotation segment moves the movable handle along a first trajectory by rotating about the first fulcrum while a relative positional relationship between the first fulcrum and the first slot is unchanged and a relative positional relationship between the second fulcrum and the second slot is changed. Rotation of the movable handle during the second rotation segment moves the movable handle along a second trajectory by rotating about the second fulcrum while a relative positional relationship between the second fulcrum and the second slot is unchanged and a relative positional relationship between the first fulcrum and the first slot is changed. Further, opening and closing the end effector occurs during the first rotation segment, but the end effector remains closed during the second rotation segment.

### Effect of the Invention

According to the treatment instrument according to the present invention, operability can be improved.

### BRIEF DESCRIPTION OF THE DRAWINGS

FIG. 1 is a diagram illustrating a treatment system according to the first embodiment.

FIG. 2 is a diagram illustrating a configuration of a treatment instrument.

FIG. 3 is a diagram illustrating a configuration of a treatment instrument.

FIG. 4 is a diagram illustrating a configuration of a handle support mechanism.

FIG. 5 is a diagram illustrating a configuration of a handle support mechanism.

FIG. 6 is a diagram illustrating a configuration of a handle support mechanism.

FIG. 7 is a diagram illustrating a configuration of a handle support mechanism.

FIG. 8 is a diagram illustrating a configuration of an opening and closing mechanism according to a second embodiment.

FIG. 9 is a diagram illustrating a configuration of a handle support mechanism according to the second embodiment.

FIG. 10 is a diagram illustrating a configuration of a handle support mechanism according to the second embodiment.

FIG. 11 is a diagram illustrating a configuration of a handle support mechanism according to the second embodiment.

FIG. 12 is a diagram illustrating a modification of the first embodiment.

### DETAILED DESCRIPTION OF THE PREFERRED EMBODIMENT(S)

### Modes for Carrying Out the Invention

Hereinafter, embodiments for carrying out the present invention (hereinafter, embodiments) will be described with reference to the accompanying drawings. Note that the present invention is not limited by the embodiments described below. In addition, in the description of the drawings, the same parts are denoted by the same reference numerals.

### First Embodiment

Schematic Configuration of the Treatment System

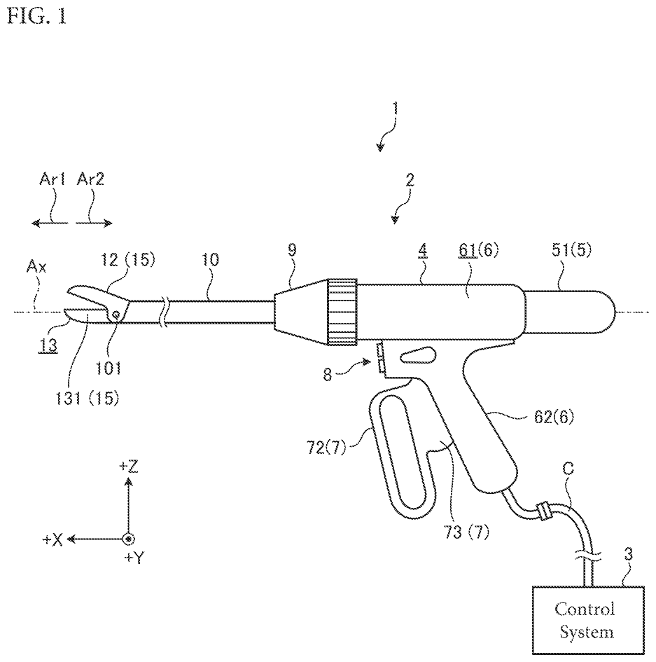

FIG. 1 is a diagram illustrating a treatment system 1 according to the first embodiment. The treatment system 1 treats the target site by imparting treatment energy to a site to be treated in a biological tissue (hereinafter, described as a target site). Note that the treatment energy in the first embodiment is ultrasonic energy and high frequency energy, but embodiments can include other procedure energies, such as thermal energy. Further, a treatment procedure that can be performed by the treatment system 1 according to the first embodiment is treatment procedure such as coagulation (sealing) of a target site, incision of a target site, or the like, but other treatment procedures can be conducted with the treatment system, such as grasping, exfoliating, and incising. In addition, treatment procedures, such as coagulation and incision, may be performed simultaneously. The treatment system 1 comprises a treatment instrument 2 and a control device 3, as shown in FIG. 1.

Configuration of the Procedure Device

In the following, in describing the configuration of the treatment instrument 2, the X-axis, Y-axis, and Z-axis are mutually orthogonal, as shown by the XYZ coordinate axis in FIG. 1. The X-axis is an axis parallel to the central axis Ax of the shaft 10 (FIG. 1), the Y-axis is an axis perpendicular to the plane of the paper, and the Z-axis is an axis along the vertical direction of FIG. 1. In addition, in the following, one side along the central axis Ax (+X-axis side) is described as a distal end side Ar1, and the other side (−X-axis side) is described as a proximal end side Ar2.

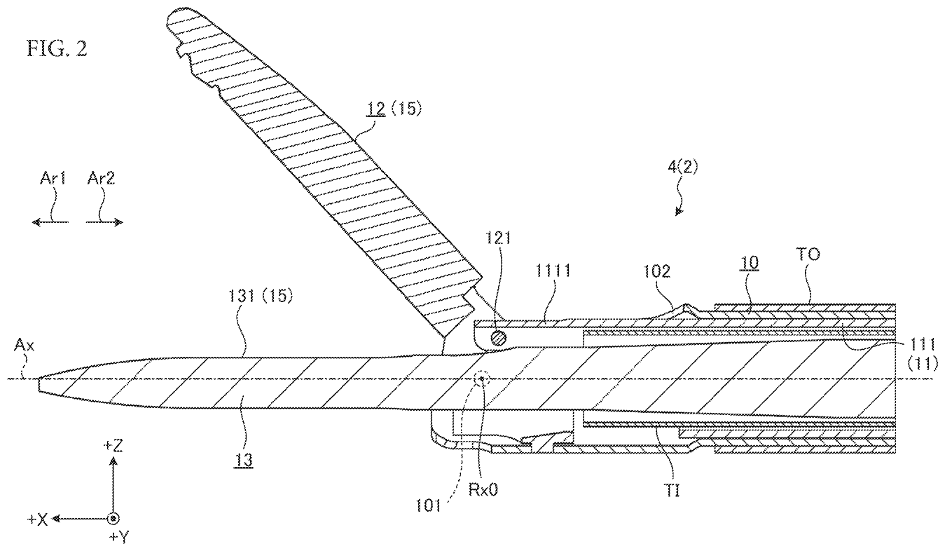

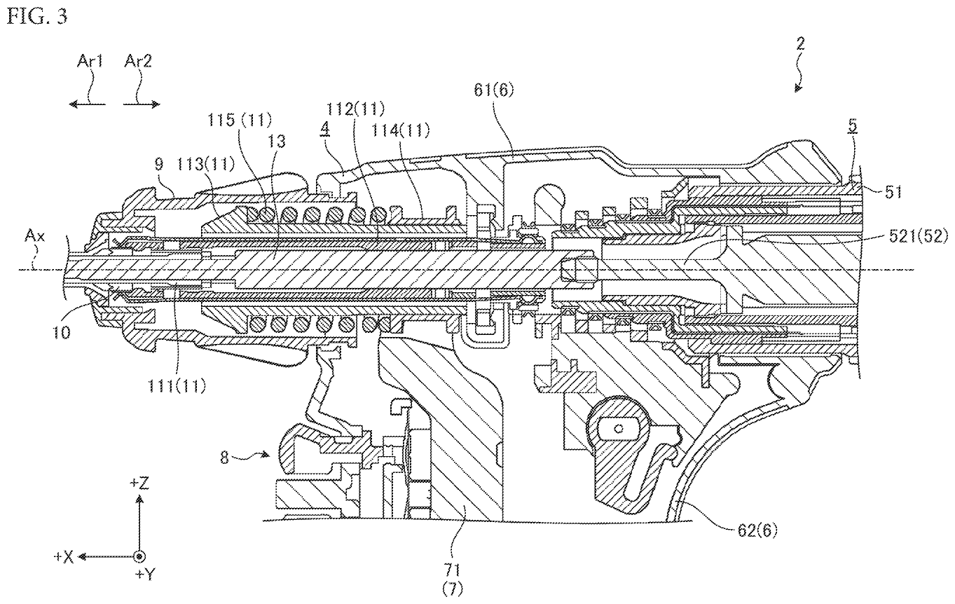

FIGS. 2 and 3 are views illustrating the configuration of the treatment instrument 2. Specifically, FIGS. 2 and 3 are cross-sectional views (as viewed from the +Y-axis side with cutting the treatment instrument 2) in the XZ plane including the central axis Ax.

The treatment instrument 2 is an ultrasonic treatment instrument which treats the target site by imparting ultrasonic energy and high frequency energy to the target site. As shown in FIGS. 1 to 3, the treatment instrument 2 comprises a handpiece 4 and an ultrasonic transducer 5.

As shown in FIGS. 1 to 3, the handpiece 4 includes a housing 6 (FIGS. 1 and 3), a movable handle 7 (FIGS. 1 and 3), a switch 8 (FIGS. 1 and 3), a rotary knob 9 (FIGS. 1 and 3), a shaft 10, an opening and closing mechanism 11 (FIGS. 2 and 3), a jaw 12 (FIGS. 1 and 2), and a vibration transmission member 13 (FIGS. 1 to 3).

The housing 6 supports the entire treatment instrument 2. As shown in FIGS. 1 and 3, the housing 6 includes a substantially cylindrical case body 61, which is coaxial with the central axis Ax and extends from the case body 61 to the −Z-axis side, and a fixed handle 62, which is grasped by an operator, such as a medical professional like a doctor or an assistant. The movable handle 7 accepts a closing operation and an opening operation, respectively, which are user operations made by the operator. Typically, in the closing operation, an operator grasps the movable handle 7, such as operation unit 72, with a finger while placing the palm of the hand on the handle body 62 to move the movable handle toward the fixed handle and, in the opening operation, an operator releases the grasping force of the operator to move the movable handle away from the fixed handle.

As shown in FIGS. 1 and 3, the movable handle 7 includes a handle base 71 (FIG. 3), an operation unit 72 (FIG. 1), and a connecting portion 73 (FIG. 1).

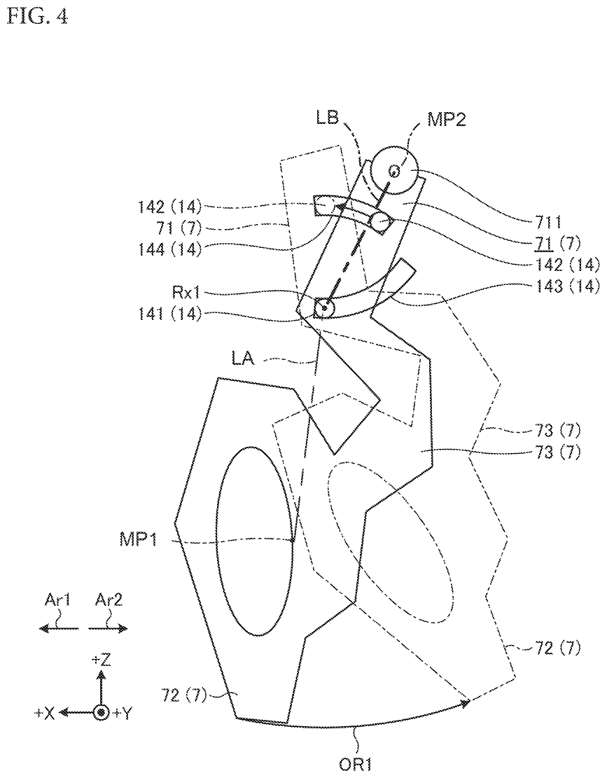

Handle base 71 is located within housing 6. The +Z-axis-side portion of the handle base portion 71 is rotatably supported to the housing 6 by the handle support mechanism 14 for rotation about a first rotational axis Rx1 and about a second rotational axis Rx2 (see FIGS. 4 to 7), each of which is parallel to the Y-axis (see FIGS. 4 to 7). Note that a detailed configuration of the handle support mechanism 14 will be described in “a configuration of a handle support mechanism” described later. Further, the +Z-axis side of the end portion of the handle base portion 71 protrudes toward the +Z-axis side, is bifurcated into branches, and functions as a transmission portion 711 (see FIGS. 4 to 7) that engages with and transmits a force to the slider 114 (part of the opening and closing mechanism 11) in accordance with the rotation of the movable handle 7. The operation unit 72 is a portion for receiving the closing operation and the opening operation by an operator such as for the closing operation and the opening operation, respectively, and is positioned outside the housing 6 as shown in FIG. 1. Connecting portion 73 is a portion for connecting the handle base 71 and the operation unit 72 and is located both inside and outside of the housing 6. When receiving the operation input by an operator for a closing operation, the movable handle 7 rotates counterclockwise around the first rotational axis Rx1 and the second rotational axis Rx2. That is, the operation unit 72 moves in a direction toward the handle body 62. On the other hand, when receiving the operation input by an operator for an opening operation, the movable handle 7 rotates clockwise around the first rotational axis Rx1 and the second rotational axis Rx2. That is, the operation unit 72 moves in a direction away from the handle body 62.

A second biasing member (not shown) is provided inside the fixed handle 62. The second biasing member is supported within the fixed handle 62 and is engaged with the connecting portion 73. The second biasing member biases the connecting portion 73 (and therefore the operation unit 72) in a direction away from the fixed handle 62. Then, because of the second biasing member, the opening control occurs by an operation of releasing the grasping force by an operator. That is and with reference to FIG. 3, when receiving an opening operation by an operator, the movable handle 7 (operation unit 72) rotates clockwise around the first rotational axis Rx1 and the second rotational axis Rx2, respectively. That is, the operation unit 72 moves in a direction away from the handle body 62.

As shown in FIGS. 1 and 3, switch 8 is provided such that a portion is exposed to the outside from the side surface of the distal end side Ar1 in the fixed handle 62. The switch 8 is operable by an operator to initiate a treatment procedure. The treatment procedure is a procedure that imparts control energy to the site of interest.

Rotary knob 9 has a substantially cylindrical shape or conical shape that is coaxial with the central axis Ax, as shown in FIG. 3, and is provided on the distal end side Ar1 of the case body 61. The rotary knob 9 accepts a rotation control, which is a user operation by an operator. By the rotation control, the rotary knob 9 rotates about the central axis Ax with respect to the case body 61. Further, in addition to the rotary knob 9 being rotatable, the jaw 12, and the vibration transmission member 13 also can be rotatable about the central axis Ax

Shaft 10 is a cylindrical pipe made of a material such as metal. Additionally, the outer peripheral surface of the shaft 10 is covered by an electrically insulating outer tube TO (FIG. 2). Further, the end portion of the distal end side Ar1 of the shaft 10 includes a first pin 101 that rotatably supports jaw 12 to rotate around the jaw rotational axis Rx0 (see FIG. 2). The first pin 101 extends in a direction perpendicular to the central axis Ax (e.g., perpendicular to the plane of the paper in FIGS. 1 and 2). Furthermore, at the end of the distal end side Ar1 of the shaft 10 and to the +Z-axis side, notches 102 extend toward the distal end side Ar1 to the proximal end side Ar2 (FIG. 2).

The jaw 12 has an opening and closing mechanism 11 that causes elements of the jaw 12 to rotate around the jaw rotational axis Rx0 in response to the opening operation and closing operation affected by manipulation of the movable handle 7 by an operator. Then, by the opening and closing mechanism, the jaw 12 opens and closes with respect to a treatment portion 131 (hereinafter, referred to as a treatment portion 131 (FIG. 2)) on the distal end side Ar1 of the vibration transmission member 13 and grasps the target site between the jaw 12 and the treatment portion 131.

As shown in FIGS. 2 and 3, the opening and closing mechanism 11 includes an inner pipe 111, a holding portion 112 (FIG. 3), a slider receiver 113 (FIG. 3), a slider 114 (FIG. 3), and a first biasing member 115 (FIG. 3). The inner pipe 111 is a cylindrical pipe having a smaller diameter than the shaft 10. Further, the inner pipe 111 is coaxial with the shaft 10 is inserted into the interior of the shaft 10. In the inner pipe 111, the +Z-axis side of the end portion of the distal end side Ar1 has an arm portion 1111 that extends toward the distal end side Ar1. The arm portion 1111 is attached to the jaw 12 by a second pin 121, which is inserted parallel to the jaw rotary shaft Rx0 (first pin 101).

Holding portion 112 is constituted by a material having an electrical insulating property, such as a resin, and has a substantially cylindrical shape. The holding portion 112 is inserted into the rotary knob 9 and the case body 61 in a state straddling the rotary knob 9 and the case body 61, as shown in FIG. 3. The holding portion 112 holds the vibration transmission member 13 inserted therein. The distal end side Ar1 of the holding portion 112 is mechanically connected to the rotary knob 9 and the shaft 10. That is, in accordance with the rotation control applied to the rotary knob 9 by an operator, the holding portion 112, the shaft 10, the jaw 12, and the vibration transmission member 13 rotates about the central axis Ax together with the rotary knob 9.

Slider receiver 113 is constituted by a material having an electrical insulating property, such as resin, and has a substantially cylindrical shape. The slider receiver 113, while the holding portion 112 is inserted therein, is movably disposed along the central axis Ax with respect to the holding portion 112. Here, the end of the distal end side Ar1 of the slider receiver 113 is connected to the end of the proximal end side Ar2 of the inner pipe 111 while the slider receiver 113 is allowed to move along the central axis Ax with respect to the holding portion 112 and the rotation around the central axis Ax of the slider receiver 113 is restricted. That is, in response to the rotation control to the rotary knob 9 by an operator, the slider receiver 113 and the inner pipe 111 rotate about the central axis Ax together with the rotary knob 9.

The slider 114 has a substantially cylindrical shape and is disposed to be movable with respect to the central axis Ax relative to the slider receiver 113. The slider 114 is engaged with the movable handle 7 (transmission portion 711) as described above.

Then, the opening/closing mechanism 11 operates as described below in response to an operation on the movable handle 7 by an operator.

In response to a closing operation applied to the movable handle 7 by an operator, slider 114 is pushed to the distal end side Ar1 along the central axis Ax by the transmission portion 711. The slider receiver 113 is subjected to a pressing force toward the distal end side Ar1 resulting from the slider 114 movement and as passed through the first biasing member 115 disposed between the slider 114 and the slider receiver 113. Further, the inner pipe 111 moves toward the distal end side Ar1 along the central axis Ax in conjunction with the slider receiver 113, causing the slider receiver 113 to move towards the distal end-side Ar1 along the central axis Ax. Further, in conjunction with the movement of the inner pipe 111, the arm portion 1111 pushes the second pin 121 toward the distal end side Ar1. Then, the jaw 12 rotates counterclockwise in FIG. 2 about the jaw rotation axis Rx0. At this time, since the second pin 121 also moves while keeping a constant distance about the jaw rotation axis Rx0, the arm portion 1111 moves to the distal end side Ar1 while deforming the +Z-axis side of notch 102. In other words, the jaw 12 moves in a direction (closing direction) proximate to the treatment portion 131. Further, in accordance with the opening operation to the movable handle 7 by an operator, the jaw 12 in FIG. 2 rotates clockwise about the jaw rotation axis Rx0. In other words, the jaw 12 moves in a direction (opening direction) spaced apart from the treatment portion 131. As described above, in response to an operation on the movable handle 7 by an operator, the jaw 12 opens and closes with respect to the treatment portion 131 and grasps the target site between the jaw 12 and the treatment portion 131.

Here, the first biasing member 115 corresponds to a biasing member according to the present invention. In FIG. 3, the first biasing member 115 is a coil spring. The first biasing member 115 acts on the slider 114 to bias the movable handle 7 in a direction to rotate clockwise (the direction spaced from the fixed handle 62) and adjusts the gripping force for gripping the target site between the jaw 12 and the treatment portion 131. More specifically, the first biasing member 115 is used to make the gripping force constant.

Jaw 12 is at least partially composed of a conductive material.

Vibration transmission member 13 is composed of a conductive material and has an elongated shape extending linearly along the central axis Ax. Further and as shown in FIG. 2, when the vibration transmission member 13 is inserted into the inner pipe 111, a treatment portion 131 projects to the outside and the proximal end side Ar2 of the vibration transmission member 13 mechanically connects to the ultrasonic transducer 5 (see FIG. 3). That is, the vibration transmission member 13 transmits the ultrasonic vibration generated by the ultrasonic transducer 5 from the proximal end side Ar2 to the treatment portion 131. In the first embodiment, the ultrasonic vibration is a longitudinal vibration vibrating in a direction along the central axis Ax. In order to ensure electrical insulation between the shaft 10 and the inner pipe 111 and the vibration transmission member 13, the outer peripheral surface of the vibration transmission member 13 is covered by an electrically insulating inner tube TI (FIG. 2).

The ultrasonic transducer 5 includes a transducer (TD) case 51 and an ultrasonic transducer 52 (FIG. 3), as shown in FIGS. 1 and 3. TD case 51 supports the ultrasonic vibrator 52 and is detachably connected to the case body 61. The ultrasonic vibrator 52 generates ultrasonic vibration under control by the control system 3. In the first embodiment, the ultrasonic vibrator 52 is constituted by a bolt-clamped Langevin transducer (BLT). In FIG. 3, for convenience of explanation, of the features of the ultrasonic vibrator 52 (BLT), FIG. 3 illustrates only the front mass 521 to be connected to the proximal end side Ar2 of the vibration transmission member 13.

Composition of the Control Device

Control system 3 collectively controls the operation of the treatment instrument 2. Specifically, the control system 3, by passing operating signals through the electrical cable C (FIG. 1), detects the operation of the switch 8 by an operator. Then, when the control system 3 detects the switch operation, operating signals and power are passed through the electric cable C to impart treatment energy to the target site grasped between the jaw 12 and the treatment portion 131. In other words, the control system 3 controls operation of the treatment instrument 2 to treat the target site. The jaws 12 and the treatment portion 131 correspond to the end effectors 15 (FIGS. 1 and 2) according to the present invention.

For example, when applying ultrasonic energy to the target site, the control system 3 supplies drive power to the ultrasonic vibrator 52 by passing it through the electrical cable C. Thus, the ultrasonic vibrator 52 generates a longitudinal vibration (ultrasonic vibration) which vibrates in a direction along the central axis Ax. The treatment portion 131 also vibrates at a desired amplitude by the longitudinal vibration. Then, an ultrasonic vibration is applied from the treatment portion 131 to the target site grasped between the jaw 12 and the treatment portion 131. In other words, ultrasonic energy is applied from the treatment portion 131 to the target site.

Further, for example, when imparting high-frequency energy to the target site, the control device 3 supplies high-frequency power between the jaw 12 and the vibration transmission member 13 by passing it through the electric cable C. Thus, a high frequency current flows through the target site grasped between the jaw 12 and the treatment portion 131. In other words, the subject site is imparted with high frequency energy.

Structure of Handle Support Mechanism

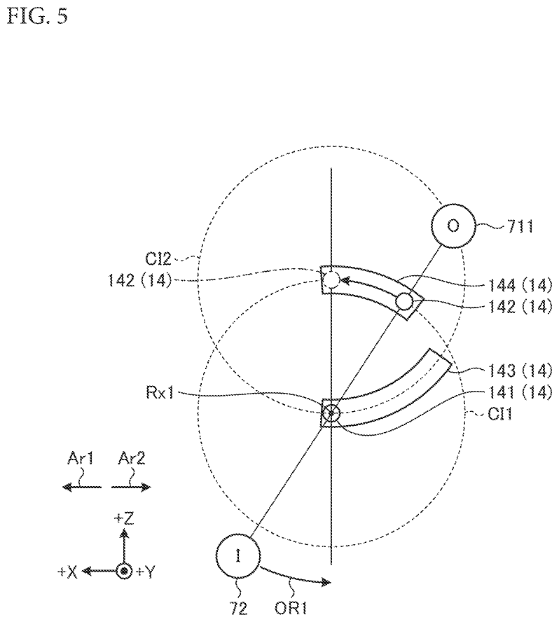

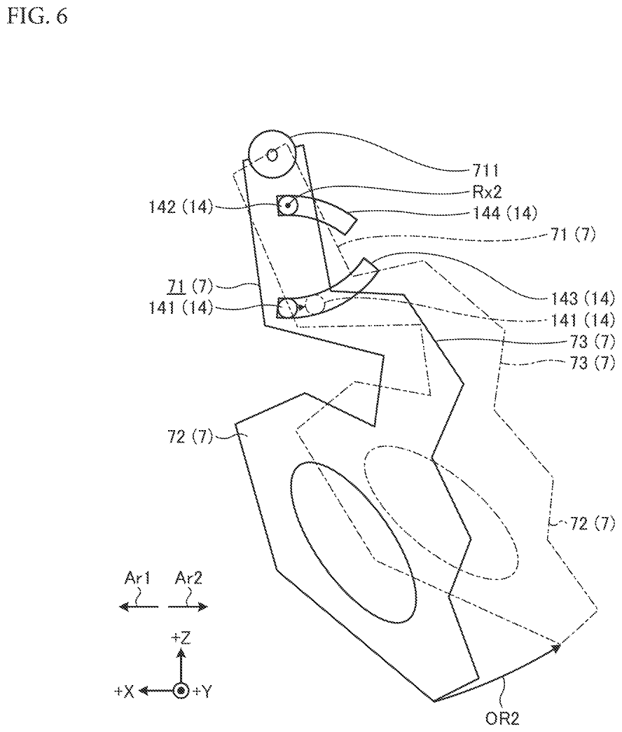

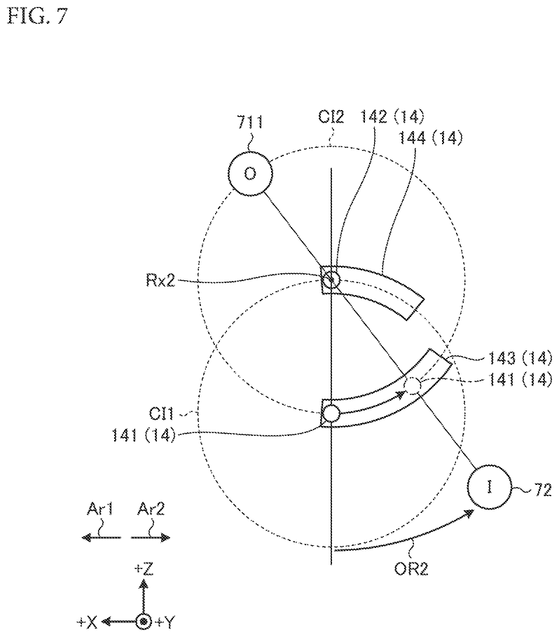

Next, a configuration of the handle support mechanism 14 will be described. FIGS. 4 to 7 are views illustrating the configuration of the handle support mechanism 14. Specifically, FIG. 4 is a diagram illustrating a moving condition depicting a first trajectory OR1 of the movable handle 7, e.g., movement along a first rotation segment. In FIG. 4, the state prior to the movement along the first trajectory OR1 is shown by a solid line, and the state after the movement along the first trajectory OR1 is shown by a dashed-dotted line. FIG. 5 is a simplified view of FIG. 4. In FIG. 5, the locations of the transmission portion 711 and the operation unit 72 correspond to the positions of those features associated with the movement along the first trajectory OR1. Also specifically, FIG. 6 is a diagram illustrating a moving condition depicting a second trajectory OR2 of the movable handle 7, e.g., movement along a second rotation segment. In FIG. 6, the state prior to the movement along the second trajectory OR2 is shown by a solid line, and the state after the movement along the second trajectory OR2 is shown by a dashed-dotted line. FIG. 7 is a simplified view of FIG. 6. In FIG. 7, the locations of the transmission portion 711 and the operation unit 72 correspond to the positions of those features associated with the movement along the second trajectory OR2. Here, in FIGS. 5 and 7, the operation unit 72 is illustrated by a circle, the letters “I” in the circle is an acronym of Input. Further, in FIGS. 4 to 7, the transmission portion 711 is illustrated by a circle, the letters “O” in the circle is an acronym of Output.

The handle support mechanism 14 comprises a first pin 141 located in a first slot 143 and a second pin 142 located in a second slot 144, as shown in FIGS. 4-7. The first pin 141 corresponds to a first fulcrum according to the present invention. In the first embodiment, the first pin 141 is provided in the handle base portion 71 on an outer surface intersecting the Y-axis, as shown in FIG. 4 and FIG. 6. The first pin 141 has a cylindrical shape extending linearly along the Y-axis. During operation, the first pin 141 functions as first rotational axis Rx1 about which the movable handle 7 rotates while the operation unit 72 moves along the first trajectory OR1. The second pin 142 corresponds to a second fulcrum according to the present invention. In the first embodiment, the second pin 142 is provided in the handle base 71 on an outer surface intersecting the Y-axis, as shown in FIG. 4 and FIG. 6. The second pin 142 has a cylindrical shape extending linearly along the Y-axis. During operation, the second pin 142 functions as second rotational axis Rx2 about which the movable handle 7 rotates while the operation unit 72 moves along the second trajectory OR2.

As shown in FIGS. 4 and 6, when viewed from the direction along the respective rotational axis Rx1, Rx2, the first and second pins 141,142 described above are disposed between the operation unit 72 and the transmission portion 711. More specifically, when viewed from a direction along the respective rotational axis Rx1, Rx2, the first pin 141 is disposed at a position spaced from the transmission portion 711 a distance that is further than the distance the second pin 142 is spaced from the transmission portion 711.

The first slot 143 is the portion through which the first pin 141 is inserted. In the first embodiment, the first slot 143 is provided on the inner surface of the housing 6 and the first slot 143 is formed by a recess in the Y-axis direction and the concave portion of the first slot 143 extends in an arc shape around the second rotational axis Rx2 corresponding to the second trajectory OR2. However, the first slot 143 is not limited to the recess on the inner surface of the housing 6 and, for example, the first slot 143 may be constituted by a hole penetrating the inside and outside of the housing 6.

The second slot 144 is the portion through which the second pin 142 is inserted. In the first embodiment, the second slot 144 is provided on the inner surface of the housing 6 and the second slot 144 is formed by a recess in the Y-axis direction and the concave portion of the second slot 144 extends in an arc shape around the first rotational axis Rx1 corresponding to the first trajectory OR1. However, the second slot 144 is not limited to the recess on the inner surface of the housing 6 and, for example, the second slot 144 may be constituted by a hole penetrating the inside and outside of the housing 6.

When receiving a closing operation by an operator, the movable handle 7 operates as described below.

First, when the movable handle 7 moves from the position most spaced apart relative to the fixed handle 62 in a direction toward the fixed handle 62, the movable handle 7 rotates about the first rotational axis Rx1 (see solid line state to dotted-dashed lines state shown in FIG. 4 and the corresponding states of FIG. 5). At this time, the second pin 142 moves longitudinally in the second slot 144 until it hits the end of the second slot 144 in the distal end side AR1 of the second slot 144, as shown in FIGS. 4 and 5. In other words, the second pin 142 moves within the second slot 144 along a portion of a circular path CI1 (see FIG. 5) about a first rotational axis Rx1. By rotating about the first rotational axis Rx1, the movable handle 7 moves along the first trajectory OR1.

Here, when the movable handle 7 moves along the first trajectory OR1, the first biasing member 115 does not act. In other words, at the time of the movement of the movable handle 7 along the first trajectory OR1, the first biasing member 115 does not bias the movable handle 7 (operation unit 72) in a direction away from the fixed handle 62. Further, at the time of the movement of the movable handle 7 along the first trajectory OR1, the second biasing member (described above) acts. In other words, when the operator or the like moves the movable handle 7 along the first trajectory OR1, the operator performs the closing operation only relative to the biasing force by the second biasing member, and the first biasing member 115 does not influence the closing operation in this state.

Also, when the movable handle 7 moves along the first trajectory OR1, the jaw 12 moves relative to the treatment portion 131 from the most open state to the closed state.

Second, after the movable handle 7 moves in the direction toward the fixed handle 62 and has completed rotation about the first rotational axis Rx1 and movement along the first trajectory OR1 (as discussed above), when the closing operation is continued by an operator, the movable handle 7 then rotates about the second rotational axis Rx2 (see solid line state to dotted-dashed lines state shown in FIG. 6 and the corresponding states of FIG. 7). At this time, the first pin 141 moves longitudinally in the first slot 143. In other words, the first pin 141 moves within the first slot 143 along a portion of a circular path CI2 (see FIG. 7) about a second rotational axis Rx2. By rotating about the second rotational axis Rx2, the movable handle 7 moves along the second trajectory OR2.

Here, when the movable handle 7 moves along the second trajectory OR2, the first biasing member 115 acts. In other words, at the time of the movement of the movable handle 7 along the second trajectory OR2, the first biasing member 115 biases the movable handle 7 (operation unit 72) in a direction away from the fixed handle 62. Further, at the time of the movement of the movable handle 7 along the second trajectory OR2, the second biasing member (described above) also acts. In other words, when the operator or the like moves the movable handle 7 along the second trajectory OR2, the operator performs the closing operation relative to the biasing force by both the first biasing member 115 and the second biasing member. Then, by the movement of the movable handle 7 along the second trajectory OR2, a gripping force corresponding to the biasing force of the first biasing member 115 is applied to the target site grip between the jaw 12 and the treatment portion 131.

During the movement of the movable handle 7, the movable handle in the handle support mechanism 14 has two different leverage ratios. The leverage ratio is defined as the ratio A/B, where A is a distance between the rotational axis and the operation unit 72 (i.e., at a midpoint of the area where the operator's force is applied) and B is a distance between the rotational axis and the transmission portion 711 (i.e., at a midpoint of the transmission point). A first leverage ratio applies to movement of the movable handle 7 along the first trajectory OR1 and a second leverage ratio applies to movement of the movable handle 7 along the second trajectory OR2A. As an example and as illustrated using FIG. 4, the midpoint of the area where the operator's force is applied is shown as MP1, the midpoint of the transmission point is shown as MP2, the distance A between the rotational axis Rx1 and the operation unit 72 at midpoint MP1 is shown as LA, the distance B between the rotational axis Rx1 and the transmission portion 711 at midpoint MP2 is shown as LB.

In the first embodiment described above, the leverage ratios are set as follows. For the first leverage ratio, the rotational axis is the first rotational axis Rx1 and first leverage ratio is the ratio of the distance between the first rotational axis Rx1 and the operation unit 72 (A1) to the distance between the first rotational axis Rx1 and the transmission portion 711 (B1) (first leverage ratio=A1/B1). For the second leverage ratio, the rotational axis is the second rotational axis Rx2 and second leverage ratio is the ratio of the distance between the second rotational axis Rx2 and the operation unit 72 (A2) to the distance between the second rotational axis Rx2 and the transmission portion 711 (B2) (second leverage ratio=A2/B2). During movement of the movable handle 7 along the first trajectory OR1, the first leverage ratio is relatively small. During movement of the movable handle 7 along the second trajectory OR1, the second leverage ratio is relatively large. Relative to each other, the second leverage ratio is larger than the first leverage ratio.

According to the first embodiment described above, the following effects can be achieved. The treatment instrument 2 according to the first embodiment includes the handle support mechanism 14 described above. Therefore, when the jaw 12 is shifted with respect to the treatment portion 131 from the most open state to the closed state (e.g., when the movable handle 7 is moved along the first trajectory OR1), the jaw 12 can be opened and closed in a large manner even if the amount of the stroke length at the time of closing the movable handle 7 is small. On the other hand, when a gripping force corresponding to the urging force of the first biasing member 115 is applied to the target site grasped between the jaws 12 and the treatment portion 131 (e.g., when the movable handle 7 is moved along the second trajectory OR2), the gripping force can be applied to the movable handle 7 with a small operating force. Accordingly, according to the treatment instrument 2 of the first embodiment, it is possible to reduce the amount of the stroke length of the movable handle 7 and to improve operability while reducing the amount of force applied by an operator to the movable handle 7.

### Second Embodiment

Next, a second embodiment will be described. In the following description, the same reference numerals will be used for the same configurations as in the first embodiment described above, and detailed description thereof will be omitted or simplified.

In the first embodiment described above, the transmission portion 711 transmitted a force toward the distal end side Ar1 with respect to the opening and closing mechanism 11 in response to the closing operation applied to the movable handle 7 by an operator. In the second embodiment, in response to the closing operation applied to the movable handle 7 by an operator, the transmission portion 711 transmits a force toward the proximal end side Ar2 with respect to the opening and closing mechanism 11. In addition, in accordance with the second embodiment, the configuration of the handle support mechanism 14 is changed. In the following, for convenience of explanation, the movable handle, the opening and closing mechanism, and the handle supporting mechanism according to the second embodiment will be described as a movable handle 7A, an opening and closing mechanism 11A, and a handle supporting mechanism 14A, respectively.

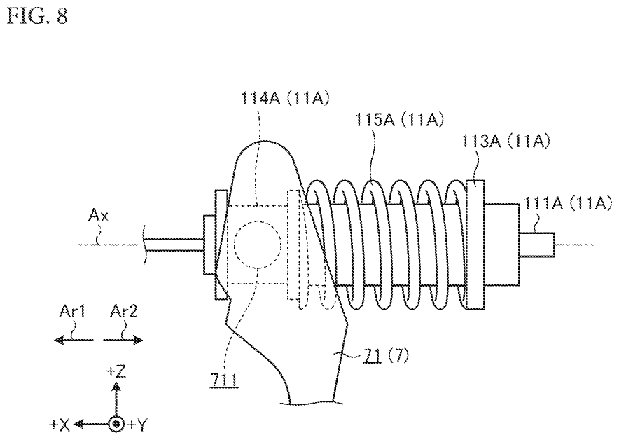

FIG. 8 is a diagram illustrating a configuration of an opening/closing mechanism 11A according to a 2 embodiment. Note that, in FIG. 8, for convenience of explanation, the illustration of the handle support mechanism 14A is omitted.

As shown in FIG. 8, the opening and closing mechanism 11A has substantially the same configuration as the opening and closing mechanism 11 described in the above-described first embodiment. In other words, the opening/closing mechanism 11A includes an inner pipe 111A, a holding portion (not shown), a slider receiver 113A, a slider 114A, and a first biasing member 115A that are each substantially the same as the corresponding component of the first biasing member 115 described in first embodiment described above.

The opening and closing mechanism 11A operates as described below in response to an operation of the movable handle 7A by an operator. In response to the closing operation of the movable handle 7A by an operator, the slider 114A is pushed toward the proximal end side Ar2 along the central axis Ax by the transmission portion 711. Further, the slider receiver 113A is subjected to a pressing force toward the proximal end side Ar2 resulting from the slider 114A movement and as passed through a first biasing member 115A disposed between the slider 114A and the slider receiver 113A. Further, the inner pipe 111A moves toward the proximal end side Ar2 along the central axis Ax in conjunction with the slider receiver 113A, causing the slider receiver 113A to move towards the proximal end-side Ar2 along the central axis Ax. Then, the jaw 12 moves in a direction (closing direction) toward the treatment portion 131 in conjunction with the movement of the inner pipe 111A. In response to the opening operation of the movable handle 7A by an operator, the jaw 12 moves in a direction (open direction) away from the treatment portion 131.

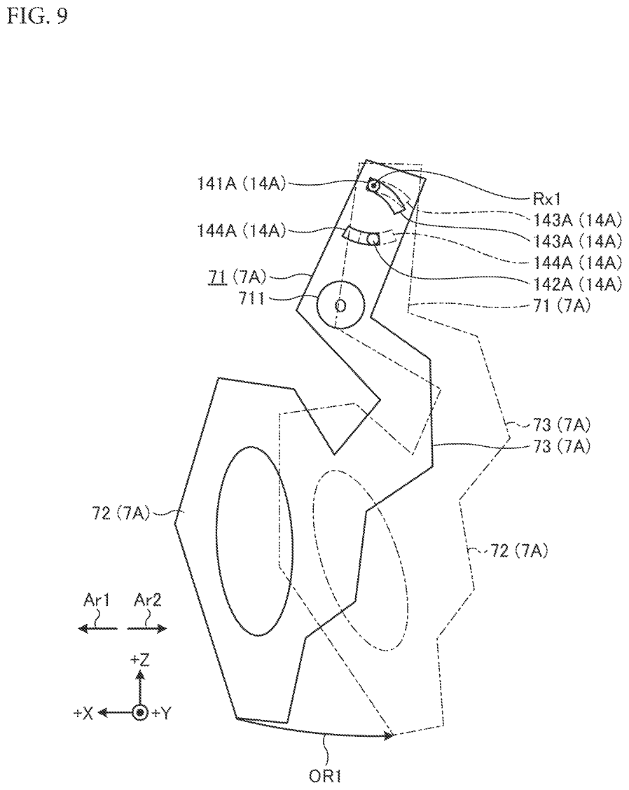

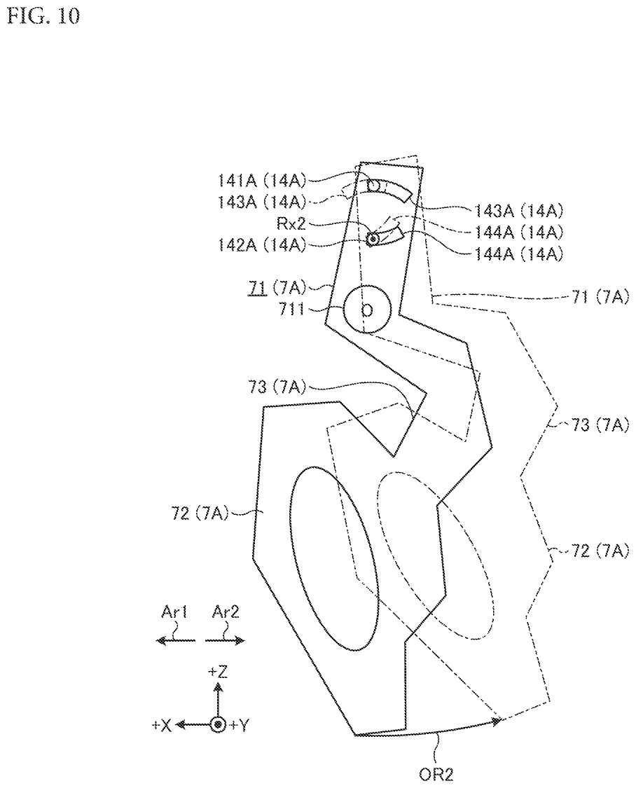

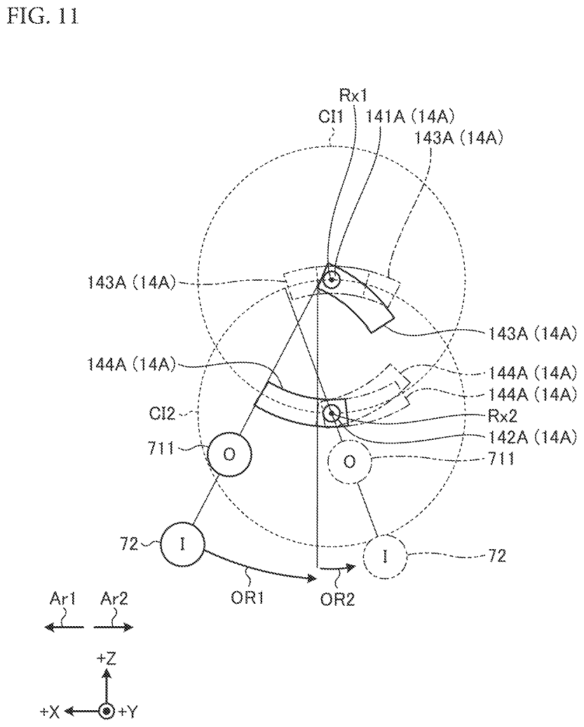

FIGS. 9 to 11 are views illustrating a configuration of a handle support mechanism 14A. Specifically, FIG. 9 is a diagram illustrating a moving condition depicting a first trajectory OR1 of the movable handle 7A, e.g., movement along a first rotation segment. In FIG. 9, the state prior to the movement along the first trajectory OR1 is shown by a solid line, and the state after the movement along the first trajectory OR1 is shown by a dashed-dotted line. Also specifically, FIG. 10 is a diagram illustrating a moving condition depicting a second trajectory OR2 of the movable handle 7A, e.g., movement along a second rotation segment. In FIG. 10, the state prior to the movement along the second trajectory OR2 is shown by a solid line, and the state after the movement along the second trajectory OR2 is shown by a dashed-dotted line. FIG. 11 is a simplified view of FIGS. 9 and 10. In FIG. 11, the locations of the transmission portion 711 and the operation unit 72 correspond to the positions of those features associated with the movement along the first trajectory OR1 and along the second trajectory OR2. Note that, in FIG. 11, the state of the movable handle before the movement along the first trajectory OR1 is shown as a solid line, the state after the movement along the first trajectory OR1 (corresponding to the state before the movement along the second trajectory OR2) is shown as a dashed-dotted line, and the state after the movement along the second trajectory OR2 is shown as a double-dashed line. In FIG. 11, the operation unit 72 is illustrated by a circle, and the letters of “I” in the circle is an acronym of Input. Further, in FIGS. 9 to 11, the transmission portion 711 is illustrated by a circle, and the letters “O” is an acronym of Output.

As shown in FIGS. 9 to 11, the handle support mechanism 14A has substantially the same configuration as the handle support mechanism 14 described in the first embodiment described above. That is, the handle support mechanism 14A includes a first pin 141A, a second pin 142A, a first slot 143A, and a second slot 144A that are substantially similar to the first pint 141, second pin 142, first slot 143, and second slot 144 described in the first embodiment 1 described above.

The first pin 141A corresponds to the first fulcrum according to the present invention. In the second embodiment, the first pin 141A is provided on the inner surface of the housing 6 and has a cylindrical shape extending linearly along the Y-axis. During operation, the first pin 141A functions as first rotational axis Rx1 about which the movable handle 7A rotates while the operation unit 72 moves along the first trajectory OR1. The second pin 142A corresponds to the second fulcrum according to the present invention. In the second embodiment, the second pin 142A is provided on the inner surface of the housing 6 and has a cylindrical shape extending linearly along the Y-axis. During operation, the second pine 142A functions as second rotational axis Rx2 about which the movable handle 7A rotates while the operation unit 72 moves along the second trajectory OR2.

As shown in FIGS. 9 to 11, when viewed from a direction along the first and second rotational axes Rx1, Rx2, the first and second pins 141A, 142A described above are disposed on a side of and spaced from both the operation unit 72 and the transmission portion 711. In other words, the operation unit 72 and the transmission portion 711 are on the same side relative to the first and second pins 141A, 142A. More specifically, when viewed from a direction along the respective rotational axis Rx1, Rx2, the first pin 141A is disposed at a position spaced from the transmission portion 711 a distance that is further than the distance the second pin 142A 141A is spaced from the transmission portion 711.

The first slot 143A is the portion through which the first pin 141A is inserted. In the second embodiment and as shown in FIGS. 9 to 11, the first slot 143A is provided in the handle base 71 and the first slot 143A is formed by a recess in the Y-axis direction on the outer surface that is intersecting the Y-axis. The concave portion of the first slot 143A extends in an arc shape around the second rotational axis Rx2. Note that the first slot 143A is not limited to a recess on the outer surface of the handle base 17 and, for example, the first slot 143A may be constituted by a hole penetrating the handle base 71 in the Y-axis direction.

The second slot 144A is the portion through which the second pin 142A is inserted. In the second embodiment and as shown in FIGS. 9 to 11, the second slot 144A is provided in the handle base 71 and the second slot 144A is formed by a recess in the Y-axis direction on the outer surface that is intersecting the Y-axis. The concave portion of the second slot 144A extends in an arc shape around the first rotational axis Rx1. Note that the second slot 144A is not limited to a recess on the outer surface of the handle base 17 and, for example, the second slot 144A may be constituted by a hole penetrating the handle base 71 in the Y-axis direction.

When receiving a closing operation by an operator, the movable handle 7A operates as described below.

First, when the movable handle 7A moves from the position most spaced apart relative to the fixed handle 62 in a direction toward the fixed handle 62, the movable handle 7 rotates about the first rotational axis Rx1 (see solid line state to dotted-dashed lines state shown in FIG. 9 and corresponding states shown in FIG. 11). At this time, the second slot 144A moves longitudinally relative to the second pin 142A that is located in the second slot 144A until the end of the second slot 144A in the distal end side AR1 of the second slot 144A contacts the second pin 142A, as shown in FIGS. 9 and 11. In other words, the second slot 144A moves along a portion of a circular path CI1 (see FIG. 11) about a first rotational axis Rx1 guided by the second pin 142A located within the second slot 144A. By rotating about the first rotational axis Rx1, the movable handle 7A moves along the first trajectory OR1.

Here, when the movable handle 7A moves along the first trajectory OR1, the first biasing member 115A does not act. In other words, at the time of the movement of the movable handle 7A along the first trajectory OR1, the first biasing member 115A does not bias the movable handle 7A (operation unit 72) in a direction away from the fixed handle 62. Further, at the time of the movement of the movable handle 7A along the first trajectory OR1, the second biasing member (described above and in the first embodiment) acts. In other words, when the operator or the like moves the movable handle 7A along the first trajectory OR1, the operator performs the closing operation only relative to the biasing force by the second biasing member, and the first biasing member 115A does not influence the closing operation in this state.

Also, when the movable handle 7A moves along the first trajectory OR1, the jaw 12 moves relative to the treatment portion 131 from the most open state to the closed state.

Second, after the movable handle 7A moves in the direction toward the fixed handle 62 and has completed rotation about the first rotational axis Rx1 and movement along the first trajectory OR1 (as discussed above), when the closing operation is continued by an operator, the movable handle 7A then rotates about the second rotational axis Rx2 (see solid line state to dotted-dashed lines state shown in FIG. 10 and corresponding states shown in FIG. 11).

At this time, the first slot 143A moves longitudinally relative to the first pin 141A that is located in the first slot 143A until the end of the first slot 143A in the distal end side AR1 of the first slot 143A contacts the first pin 141A, as shown in FIGS. 10 and 11. In other words, the first slot 143A moves along a portion of a circular path CI2 (see FIG. 11) about a second rotational axis Rx2 guided by the first pin 141A located within the first slot 143A. By rotating about the second rotational axis Rx2, the movable handle 7A moves along the second trajectory OR2.

Here, when the movable handle 7A moves along the second trajectory OR2, the first biasing member 115A acts. In other words, at the time of the movement of the movable handle 7A along the second trajectory OR2, the first biasing member 115A biases the movable handle 7A (operation unit 72) in a direction away from the fixed handle 62. Further, at the time of the movement of the movable handle 7A along the second trajectory OR2, the second biasing member (described above and in the first embodiment) also acts. In other words, when the operator or the like moves the movable handle 7A along the second trajectory OR2, the operator performs the closing operation relative to the biasing force by both the first biasing member 115A and the second biasing member. Then, by the movement of the movable handle 7A along the second trajectory OR2, a gripping force corresponding to the biasing force of the first biasing member 115A is applied to the target site grip between the jaw 12 and the treatment portion 131.

Similar to the first embodiment, the movable handle 7A in the handle support mechanism 14A in the second embodiment has two different leverage ratios. Also, in the second embodiment, the leverage ratio at the time of movement of the movable handle 7A along the first and second trajectories OR1, OR2 is defined in the same manner as in the first embodiment described above.

Specifically, the rotational axis at the time of movement of the movable handle 7A along the first track OR1 is a first rotational axis Rx1. Therefore, the leverage ratio at the time of the movement of the movable handle 7A along the first trajectory OR1 (hereinafter, described as the first leverage ratio) is a ratio of the distance between the first rotational axis Rx1 and the operation unit 72 (A3) to the distance between the first rotational axis Rx1 and the transmission section 711 B3) (first leverage ratio=A3/B3). During movement of the movable handle 7A along the first trajectory OR1, the first leverage ratio is relatively small. Additionally, the rotational axis at the time of moving the movable handle 7A along the second track OR2 is a second rotational axis Rx2. Therefore, the leverage ratio at the time of movement of the movable handle 7A along a second trajectory OR2 (hereinafter, described as the second leverage ratio) is the ratio of the distance between the second rotational axis Rx2 and the operating unit 72 (A4) to the distance between the second rotational axis Rx2 and the transmission portion 711 (B4) (second leverage ratio=A4/B4). During movement of the movable handle 7A along the second trajectory OR1, the second leverage ratio is relatively large. Relative to each other, the second leverage ratio is larger than the first leverage ratio.

Even when the structure (the movable handle 7A, the opening and closing mechanism 11A, and the handle supporting mechanism 14A) according to the second embodiment described above is employed, the same effect as in the first embodiment described above is achieved.

### Other Embodiments

While embodiments for carrying out the present invention have been described above, the present invention should not be limited only by the first and second embodiments described above.

In the first embodiment described above, the first and second pins 141,142 may each be provided in the housing 6 and the first and second slots 143, 144 may be provided in the movable handle 7. Similarly, in the second embodiment described above, the first and second pins 141A, 142A may be provided on the movable handle 7A and the first and second slots 143A, 144A may be provided on the housing 6. In further alternatives, the one pin can be provided on the housing and its corresponding slot may be provided on the movable handle and the other pin can be provide on the movable handle and its corresponding slot may be provided on the housing.

In the first and second embodiments described above, as the treatment instrument according to the present invention, a configuration is set to impart both ultrasonic energy and high frequency energy to a target site, but the present invention is not limited thereto. As the treatment instrument according to the present invention, it may be employed a configuration that imparts at least one treatment energy to a target site and the treatment energy may be at least any one of ultrasonic energy, high frequency energy, and thermal energy. Here, “imparting heat energy to a target site” means transmitting heat generated in a heater or the like to a target site.

In the first and second embodiments described above, the biasing member according to the present invention (the first biasing member 115 and 115A) is not limited to a coil spring, and any member having elasticity may be employed as the biasing member.

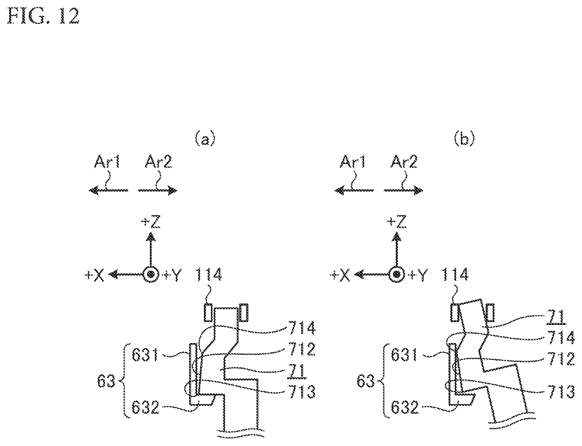

FIG. 12 is a diagram illustrating a modification of the first embodiment. In the first embodiment described above, instead of the handle support mechanism 14, the structure according to the present modification shown in FIG. 12 may be employed. Specifically and as shown in FIG. 12, the contact portion 63 can be provided in the inner surface of the housing 6 and in a position on the +X-axis side with respect to the handle base 71. As shown in FIG. 12, contact portion 63 comprises an abutment body 631 and a receiving portion 632. The abutment body 631 is a portion where the handle base 71 abuts. In this modification, the abutment body 631 is constituted by a flat plate which is substantially parallel to the YZ plane. Receiving portion 632 is a portion of the contact body 63 extending toward the proximal end side Ar2 from the end of the −Z-axis side of the abutment body 631. In this modification, the receiving portion 632 is constituted by a flat plate which is substantially parallel to the XY plane.

An opposing surface 712 facing the contact portion body 631 is provided in the handle base 71 on the side surface of the distal end side Ar1. In this modification, the opposing surface 712 is constituted by a flat surface. In the following, in the opposing surface 712, the edge portion of the −Z-axis side is described as a first fulcrum 713 and the edge portion of the +Z-axis side is described as a second fulcrum 714. The first and second fulcrums 713, 714 are located on the −Z-axis side of the handle base 71, rather than the transmission portion 711 that engages the slider 114. In this modification, the first and second fulcrums 713, 714 are constituted by a straight line extending along the Y-axis.

The movable handle 7 according to the present modification operates as described below in response to a closing operation of the movable handle 7 by an operator. First, the movable handle 7 rotates about the first fulcrum 713 until only the first fulcrum 713 of the first and second fulcrums 713,714 abuts against the abutment body 631 and the second fulcrum 714 abuts against the abutment body 631 when moving in a direction proximate to the fixed handle 62 from a position most spaced apart relative to the fixed handle 62 (the state shown in (a) of FIG. 12). By rotating about the first fulcrum 713, the movable handle 7 moves along a first trajectory.

Here, when the movable handle 7 moves along the first trajectory, the first biasing member 115 does not act. In other words, at the time of the movement of the movable handle 7 along the first trajectory, the first biasing member 115 does not bias the movable handle 7 (operation unit 72) in a direction away from the fixed handle 62. Further, at the time of the movement of the movable handle 7 along the first trajectory, the second biasing member (described in the first embodiment described above) acts. That is, when the operator or the like moves the movable handle 7 along the first trajectory, the operator performs the closing operation only relative to the biasing force by the second biasing member, and the first biasing member 115 does not influence the closing operation in this state.

Then, when the movable handle 7 moves along the first trajectory, the jaw 12 moves relative to the treatment portion 131 from the most open state to the closed state.

Second, after moving the movable handle 7 in a direction toward the fixed handle 62 along the first trajectory, when the closing operation is continued by an operator (as shown in (b) in FIG. 12), only the second fulcrum 714 of the first and second fulcrums 713,714 abuts against the contact portion body 631 and the movable handle 7 rotates around the second fulcrum 714. By rotating about the second fulcrum 714, the movable handle 7 moves along a second trajectory. Here, when the movable handle 7 moves along the second trajectory, the first biasing member 115 acts. In other words, at the time of the movement of the movable handle 7 along the second trajectory, the first biasing member 115 biases the movable handle 7 (operation unit 72) in a direction away from the fixed handle 62. Further, at the time of this movement, the second biasing member (described above) acts. In other words, when the operator or the like moves the movable handle 7 along the second trajectory, the operator performs the closing relative to the biasing force by both the first biasing member 115 and the second biasing member. Then, by the movement of the movable handle 7 along the second trajectory, a gripping force corresponding to the biasing force of the first biasing member 115 is applied to the target site grip between the jaw 12 and the treatment portion 131.

Similar to the first embodiment and the second embodiment, the movable handle 7 in the above modification has two different leverage ratios. Also, in the modification described above, the leverage ratio at the time of the movement of the movable handle 7 along the first and second trajectories is defined in the same manner as in the first embodiment described above.

Specifically, the rotational axis at the time of movement of the movable handle 7 along the first trajectory is the first fulcrum 713. Therefore, the leverage ratio at the time of the movement of the movable handle 7 along the first trajectory (hereinafter, described as the first leverage ratio) is a ratio of the distance between the first fulcrum 713 and the operation unit 72 (A5) to the distance between the first fulcrum 713 and the transmission portion 711 (B5) (first leverage ratio=A5/B5). In the modification, during movement of the movable handle 7 along the first trajectory, the first leverage ratio is relatively small.

Additionally, the rotational axis at the time of movement of the movable handle 7 along the second trajectory is the second fulcrum 714. Therefore, the leverage ratio at the time of the movement of the movable handle 7 along the second trajectory (hereinafter, described as the second leverage ratio) is a ratio of the distance between the second fulcrum 714 and the operation unit 72 (A6) to the distance between the second fulcrum 714 and the transmission section 711 (B6) (second leverage ratio=A6/B6). In the modification, during movement of the movable handle 7 along the second trajectory, the second leverage ratio is relatively large. In the modification, relative to each other, the second leverage ratio is larger than the first leverage ratio.

Even when the structure according to the present modification described above is employed, the same effect as in the first embodiment described above is achieved.

### DESCRIPTION OF SYMBOLS

1 Treatment system
2 Treatment instrument
3 Control system
4 Handpiece
5 Ultrasonic transducer
6 Housing
7, 7A movable handle
8 Switch
9 Rotary knob
10 Shaft
11, 11A Opening and closing mechanism
12 Jaw
13 Vibration transmission member
14, 14A handle support mechanism
15 End effector
51 TD case
52 Ultrasonic vibrator
61 Case body
62 Fixed handle
63 Contact portion
71 Handle base
72 Operation unit
73 Connecting portion
101 First pin
102 Notch
111, 111A inner pipe
112 Holding portion
113, 113A slider receiver
114, 114A slider
115, 115A first biasing member
121 Second pin
131 Treatment portion
141, 141A first pin
142, 142A second pin
143, 143A first slot
144, 144A second slot
521 Front mass
631 Abutment body
632 Receiving portion
711 Transmission portion
712 Opposing surface
713 First fulcrum
714 Second fulcrum
1111 Arm portion
Ar1 distal end side
Ar2 proximal end side
Ax central axis
C Electrical cable
CI1, CI2 circle
OR1 first trajectory
OR2 second trajectory
Rx0 jaw rotation axis
Rx1,Rx2 first and second rotational axes
TI inner tube
TO Outer tube

What is claimed is:

1. A treatment instrument, comprising:
a housing having a fixed handle configured to be grasped by a user;
an end effector configured to grasp a biological tissue by opening and closing;
a movable handle rotatable relative to the housing by moving in a direction toward the fixed handle and away from the fixed handle;
an opening and closing mechanism configured to open and close the end effector in response to a rotation of the movable handle;
a first fulcrum inserted in a first slot, the first fulcrum located at a first location and serving as a first rotation axis for rotating the movable handle; and
a second fulcrum inserted in a second slot, the second fulcrum located at a second position and serving as a second rotation axis for rotating the movable handle,
wherein the first location is different from the second location.

2. The treatment instrument according to claim 1, wherein the movable handle is movable along a first trajectory by rotating about the first fulcrum while a relative positional relationship between the second fulcrum and the second slot is changed, and the movable handle is movable along a second trajectory by rotating about the second fulcrum while a relative positional relationship between the first fulcrum and the first slot is changed.

3. The treatment instrument according to claim 1, the movable handle comprises:
an operation unit configured to accept a rotation operation by the user; and
a transmission unit configured to transmit a force to the opening and closing mechanism in response to the rotation of the movable handle,
wherein, when viewed from a direction along the first rotation axis:
the first fulcrum and the second fulcrum are located between the operation unit and the transmission unit, and
a first distance between the first fulcrum and the transmission unit is larger than a second distance between the second fulcrum and the transmission unit.

4. The treatment instrument according to claim 1, the movable handle comprising:
an operation unit configured to accept a rotation operation by the user; and
a transmission unit configured to transmit a force to the opening and closing mechanism in response to the rotation of the movable handle,
wherein, when viewed from a direction along the rotation axis
the first fulcrum and the second fulcrum are disposed on a same side relative to the operation unit and the transmission unit, and
a first distance between the first fulcrum and the transmission unit is larger than a second distance between the second fulcrum and the transmission unit

5. The treatment instrument according to claim 1, wherein, when the movable handle moves in the direction toward the fixed handle from a position most separated from the fixed handle, the movable handle is movable along a first trajectory by rotating about the first fulcrum while a relative positional relationship between the second fulcrum and the second slot is changed.

6. The treatment instrument according to claim 5, wherein after completing movement along the first trajectory and then the movable handle moves in a direction further proximate to the fixed handle, the second fulcrum strikes an end of the second slot, and the movable handle is movable along a second trajectory by rotating about the second fulcrum while a relative positional relationship between the first fulcrum and the first slot is changed.

7. The treatment instrument according to claim 2, wherein the first slot has a first arc shape corresponding to the second trajectory.

8. The treatment instrument according to claim 2, wherein the second slot has a second arc shape corresponding to the first trajectory.

9. The treatment instrument according to claim 2, wherein the opening and closing mechanism includes:
a biasing member configured to adjust a gripping force by which the end effector grips the biological tissue;
wherein the biasing member does not act when the movable handle moves along the first trajectory, and the biasing member acts when the movable handle moves along the second trajectory.

10. The treatment instrument according to claim 1, wherein the end effector treats the biological tissue by imparting a treatment energy to the biological tissue.
